(19) United States
(12) Patent Application Publication
(10) Pub. No.: US 2015/0250988 A1
(43) Pub. Date: Sep. 10, 2015

(54) Prolate Spheroid-Shaped Balloon

(71) Applicant: Translational Biologic Infusion Catheter, LLC, Gilbert, AZ (US)

(72) Inventors: Nabil Dib, Paradise Valley, AZ (US); Alan Iranian, Maple Grove, MN (US)

(73) Assignee: Translational Biologic Infusion Catheter, LLC, Gilbert, AZ (US)

(21) Appl. No.: 14/201,495

(22) Filed: Mar. 7, 2014

(51) Int. Cl. A61M 25/10

(52) U.S. Cl. CPC A61M 25/104; A61M 25/1002; A61M 25/10181; A61M 2210/12

(57) ABSTRACT

A balloon system is disclosed for positioning a distal end of a catheter at a treatment site. The system includes an elongated catheter shaft that is formed with a lumen and a tubular shaped balloon membrane that is made of a compliant material. For the system, the proximal and distal ends of the balloon membrane are affixed to an outer surface of the shaft to establish an inflation chamber between the balloon membrane and the outer surface of the shaft. The balloon membrane can have a non-uniform thickness between the proximal and distal ends of the membrane to establish a selected membrane shape when the balloon is inflated. For example, the selected membrane shape can be a prolate spheroid. With this arrangement, a relatively short and a relatively flat inter-contact surface in the midway region of the membrane is obtained when the balloon is inflated.

### FIELD OF THE INVENTION

The present invention pertains generally to catheters having an inflatable balloon that can be used to position the distal end of the catheter at a target site in the vasculature of a patient. More particularly, the present invention pertains to a balloon for a balloon catheter that provides minimal radial forces between the balloon and a vessel wall when inflated to decrease the incidence of vessel dissection and perforation. The present invention is particularly, but not exclusively, useful as a balloon that can adapt to different vessel diameters to minimize the need for multiple balloon catheters.

### BACKGROUND OF THE INVENTION

Inflatable balloons are often used to dilate a blockage in an artery with minimal radial forces on the arterial wall. This is done to cause less vascular injury such as dissection and perforation. Also, balloons can be employed for placing stents in the vasculature of a patient. In another application, balloons can be used to anchor a portion of a catheter at a target site in the vasculature of a patient. Typically, for this purpose, an inflatable balloon is mounted at the distal end of the catheter. The distal end of the catheter is then inserted into the patient and advanced within the patient's vasculature to a treatment site. There, at the treatment site, the balloon is inflated until it contacts the wall of the vessel. Once positioned, the catheter can be used, for example, to perform diagnostic imaging, infusion of a medicament, the placement of a stent, or to anchor the catheter as required by a particular protocol.

Generally, for these procedures, balloons are made of a compliant material. In more detail, balloons made of a compliant material continue to expand as the internal pressure in the balloon is increased. This is to be contrasted with a non-compliant balloon which expands to a predetermined size and shape as the internal pressure in the balloon is increased. In one application, a non-compliant balloon can be used to exert force on a vessel wall, for example, to expand a constricted artery.

Heretofore, compliant balloons have been used which, when inflated, establish a substantially tubular, ‘hot dog’ shape within a vessel. With increasing inflation, the hot dog shaped balloons elongate, increasing the contact area between the balloon and the internal wall of the vessel. This results in a substantial contact area between the balloon and internal vessel wall. In some cases, however, a substantial contact area between the balloon and internal vessel wall is undesirable. Moreover, it may be undesirable to have a balloon/vessel wall contact area that varies with inflation pressure.

In light of the above, it is an object of the present invention to provide a balloon for a catheter that can operationally adapt to different vessel diameters and tolerate high-pressure inflation within the vasculature of a patient. Another object of the present invention is to provide a balloon for a catheter that maintains a substantially constant inter-contact surface area between the balloon and a vessel wall over a range of inflation pressures. Yet another object of the present invention is to provide a prolate spheroid-shaped balloon that is easy to use, is simple to implement and is comparatively cost effective.

### SUMMARY OF THE INVENTION

In accordance with the present invention, a balloon system for positioning a distal end of a catheter at a treatment site includes an elongated catheter shaft that is formed with a lumen. For the balloon system, the shaft defines a longitudinal axis, extends from a proximal end to a distal end, and has an outer diameter dₒ.

In addition to the shaft, the system includes a tubular shaped balloon membrane that is made of a compliant material such as urethane. Typically, the balloon membrane has a length L between its proximal end and its distal end. In any event, the actual value for the length L is discretionary and will depend on the particular application. For the system, the proximal and distal ends of the balloon membrane are affixed to an outer surface of the shaft to establish an inflation chamber between the balloon membrane and the outer surface of the shaft.

For the present invention, the balloon membrane can have a non-uniform thickness between the proximal and distal ends of the membrane to establish a selected membrane shape when the balloon is inflated. For example, the selected membrane shape can be a prolate spheroid.

In one embodiment of the balloon system, the balloon membrane can be thicker at the ends (i.e. the proximal and distal ends) than a region midway between the ends. With this arrangement, a relatively short and a relatively flat inter-contact surface in the midway region of the membrane is obtained when the balloon is inflated. In more detail, the balloon membrane can have a central thickness t, in the region midway between the proximal and distal membrane ends and a membrane thickness tₑ at the proximal and distal membrane ends, with tₑ >t_{c}.

Also for the balloon system, an inflation unit is included to inflate the balloon. For example, an inflation lumen can be formed in the catheter shaft to establish fluid communication between the inflation unit and the inflation chamber of the balloon.

During an inflation of the balloon by an inflation pressure Pᵢ, a radial distance r_{c} is established from the outer surface of the shaft to the inter-contact surface of the midway region. In addition, for the balloon system of the present invention, the radial distance r_{c} varies proportionally with changes in Pᵢ inside the inflation chamber. Typically, the radial distance r_{c} will be as required by the application. For example, it will usually be less than about 35 mm with a balloon inflation pressure Pᵢ less than about 15 atmospheres. In one embodiment, a balloon is designed to be inflated up to 14 atm of pressure.

In one aspect of the present invention, the balloon membrane is designed such that sequential configurations of the balloon membrane during an inflation cycle present a substantially same area for the inter-contact surface of the midway region. For example, this functionality can be achieved by controlling the thickness between the proximal and distal ends of the membrane during the balloon membrane manufacturing process.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a schematic/perspective view of the balloon system of the present invention;

FIG. 2 is a cross-section view of a portion of the balloon system as seen along the line 2-2 in FIG. 1, shown with the balloon inflated by an inflation pressure Pᵢ;

FIG. 3 is a cross-section view of a portion of the balloon system as seen along the line 2-2 in FIG. 1, shown with the balloon inflated by an inflation pressure Pᵢ together with two other balloon configurations (shown by dotted lines) corresponding to two other inflation pressures; and

FIG. 4 is graph showing a balloon inflation pressure (ordinate) as a function of radial distance r_{c} from the outer surface of the shaft to the inter-contact surface of the midway region (abscissa).

### DESCRIPTION OF THE PREFERRED EMBODIMENTS

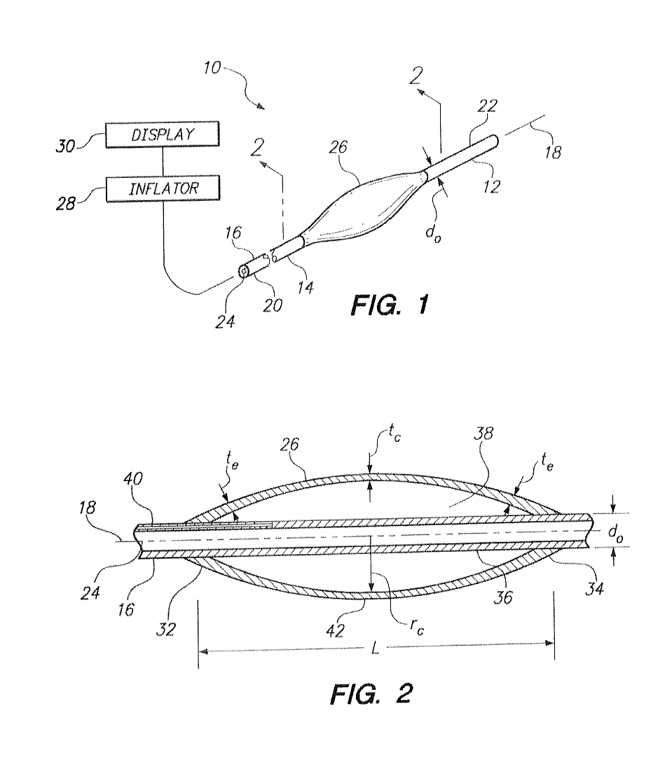

Referring initially to FIG. 1 a balloon system in accordance with the present invention is shown and is generally designated 10. In one application, the balloon system 10 can be used to position a distal end 12 of a catheter 14 at a treatment site within the vasculature of a patient (not shown). FIG. 1 also shows that the balloon system 10 includes a shaft 16 that defines a longitudinal axis 18, extends from a proximal end 20 to a distal end 22, and has an outer diameter dₒ. FIG. 1 also shows that the shaft 16 is formed with a lumen 24.

Continuing with FIG. 1, it can be seen that the balloon system 10 also includes a tubular shaped balloon membrane 26. Typically, for the present invention, the balloon membrane 26 is made of a compliant material such as urethane. FIG. 1 also shows that the balloon system 10 can include an inflator 28 that is operationally connected to the proximal end 20 of the shaft 16 to selectively inflate the balloon. Also, as shown, a display 30 can be operationally connected to the inflator 28 to provide information, such as inflation pressure, to a user (not shown), such as a physician, during a balloon inflation.

FIG. 2 shows that the balloon membrane 26 has a length L between its proximal end 32 and its distal end 34 and, typically, L will be between about 8-35 mm for use in the coronary and between about 20-150 mm for use in the peripheral arteries. It can also be seen in FIG. 2 that the proximal end 32 and distal end 34 of the balloon membrane 26 are affixed to an outer surface 36 of the shaft 16. With this cooperative structural arrangement, an inflation chamber 38 is established between the balloon membrane 26 and the outer surface 36 of the shaft 16. Also, FIG. 2 shows that the shaft 16 can be formed with an inflation lumen 40 to establish fluid communication between the inflator 28 (see FIG. 1) and the inflation chamber 38.

Continuing with reference to FIG. 2, it can be seen that the balloon membrane 26 can be thicker at the ends (i.e. the proximal end 32 and distal end 34) than a region 42 that is midway between the proximal end 32 and distal end 34. As shown, the balloon membrane 26 can have a central thickness t_{c} in the region 42 midway between the proximal end 32 and distal end 34 and a membrane thickness tₑ at the proximal end 32 and distal end 34, with tₑ >t_{c}. This arrangement allows for a relatively short and a relatively flat inter-contact surface in the midway region 42 of the membrane 26 to be obtained when the balloon is inflated. FIG. 2 illustrates that the balloon membrane 26 can have a non-uniform thickness between the proximal end 32 and distal end 34 to establish a selected membrane shape when the balloon is inflated. For the embodiment shown in FIG. 2, the selected membrane shape is a prolate spheroid. FIG. 2 shows the balloon inflated to an inflation pressure Pᵢ. As shown, at the inflation pressure Pᵢ, the midway region 42 of the membrane 26 is spaced at a radial distance r_{c} from the axis 18 of the shaft 16.

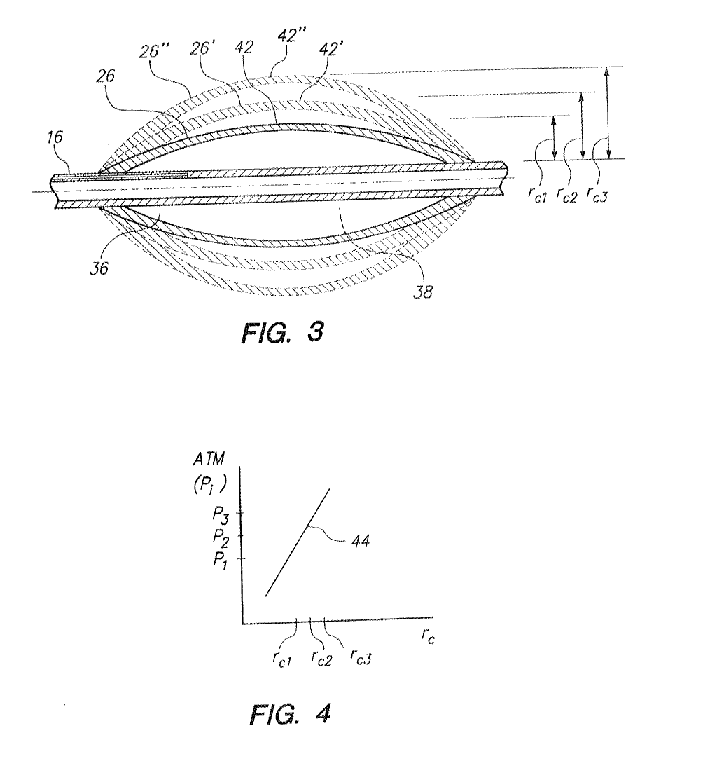

FIGS. 3 and 4 illustrate that the radial distance between the midway region 42 of the membrane 26 and the outer surface 36 of the shaft 16 varies proportionally with changes in Pᵢ inside the inflation chamber 38. Specifically, FIG. 3 shows the membrane 26 at an inflation pressure P₁ has a radial distance r_{c1} between the midway region 42 of the membrane 26 and the outer surface 36 of the shaft 16. At an inflation pressure P₂, with P₂>P₁, membrane 26′ has a radial distance r_{c2}, with r_{c2}>r_{c1}, between the midway region 42′ of the membrane 26′ and the outer surface 36 of the shaft 16. Also, at an inflation pressure P₃, with P₃>P₂, membrane 26″ has a radial distance r_{c3}, with r_{c3}>r_{c2}, between the midway region 42″ of the membrane 26″ and the outer surface 36 of the shaft 16. FIG. 3 also illustrates that the balloon membrane 26 is designed such that sequential configurations of the balloon membrane 26 during an inflation cycle present a substantially same area for the inter-contact surface of the midway region 42. FIG. 4 shows a plot 44 of balloon inflation pressure (ordinate) as a function of radial distance r_{c} from the outer surface 36 (FIG. 3) of the shaft 16 to the inter-contact surface of the midway region 42 (abscissa). From FIG. 4, it can be seen that the radial distance r_{c} between the midway region 42 (FIG. 3) of the membrane 26 and the axis 18 of the shaft 16 varies proportionally with changes in P, inside the inflation chamber 38.

While the particular prolate spheroid-shaped balloon as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A system which comprises:
an elongated shaft formed with a lumen, wherein the shaft defines a longitudinal axis, has a proximal end and a distal end, and has an outer diameter dₒ;
a tubular shaped balloon membrane having a proximal end affixed to an outer surface of the shaft and a distal end affixed to the outer surface of the shaft to establish an inflation chamber between the balloon membrane and the outer surface of the shaft, wherein the balloon membrane has a central thickness t_{c} in a region midway between the proximal and distal ends of the membrane, with a membrane thickness tₑ at the proximal end of the membrane, and a substantially same membrane thickness tₑ at the distal end of the membrane, and wherein tₑ>t_{c} to form a membrane capable of operationally adapting to different vessel diameters when the balloon is inflated in a vessel; and
an inflation unit connected in fluid communication with the inflation chamber of the balloon to inflate the balloon.

2. A system as recited in claim 1 wherein during an inflation of the balloon, a radial distance r_{c} from the outer surface of the shaft to the inter-contact surface of the midway region, is established by an inflation pressure Pᵢ inside the inflation chamber.

3. A system as recited in claim 2 wherein r_{c} varies proportionally with changes in Pᵢ inside the inflation chamber.

4. A system as recited in claim 2 wherein the radial distance r_{c} is less than 35 mm.

5. A system as recited in claim 1 wherein the inflation pressure Pᵢ is less than about 15 atmospheres.

6. A system as recited in claim 1 wherein the balloon membrane has a length L between the proximal end and the distal end, and L is less than 150 mm.

7. A system as recited in claim 1 wherein the balloon membrane is made of a compliant material.

8. A system as recited in claim 7 wherein the compliant material is urethane.

9. A system as recited in claim 1 wherein sequential configurations of the balloon membrane during an inflation cycle present a substantially same area for the inter-contact surface of the midway region.

10. A system which comprises:
an elongated shaft formed with a lumen;
a tubular shaped balloon membrane having a proximal end affixed to an outer surface of the shaft and a distal end affixed to the outer surface of the shaft to establish an inflation chamber between the balloon membrane and the outer surface of the shaft, wherein the balloon membrane is made of a compliant material and has a non-uniform thickness between the proximal and distal ends of the membrane to establish a selected membrane shape when the balloon is inflated; and
an inflation unit connected in fluid communication with the inflation chamber of the balloon to inflate the balloon.

11. A system as recited in claim 10 wherein the selected membrane shape is a prolate spheroid.

12. A system as recited in claim 10 wherein the balloon membrane has a central thickness t_{c} in a region midway between the proximal and distal ends of the membrane, with a membrane thickness tₑ at the proximal end of the membrane, and a substantially same membrane thickness tₑ at the distal end of the membrane, and wherein tₑ>t_{c}.

13. A system as recited in claim 12 wherein during an inflation of the balloon, a radial distance r_{c} from the outer surface of the shaft to the inter-contact surface of the midway region is established by an inflation pressure Pᵢ inside the inflation chamber and wherein r_{c} varies proportionally with changes in Pᵢ inside the inflation chamber.

14. A system as recited in claim 10 wherein the radial distance r_{c} is less than 35 mm with an inflation pressure Pᵢ less than 15 atmospheres.

15. A system as recited in claim 10 wherein the balloon membrane has a length L between the proximal end and the distal end, and L is less than 150 mm.

16. A system as recited in claim 10 wherein the balloon membrane is made of a compliant material.

17. A system as recited in claim 10 wherein sequential configurations of the balloon membrane during an inflation cycle present a substantially same area for the inter-contact surface of the midway region.

18. A method for positioning a distal end of an elongated catheter shaft at a treatment site, the method comprising the steps of:
providing an elongated catheter shaft formed with a lumen, wherein the shaft defines a longitudinal axis, has a proximal end and a distal end, and has an outer diameter dₒ; affixing a proximal end of a tubular shaped balloon membrane to an outer surface of the shaft;
affixing a distal end of the balloon membrane to the outer surface of the shaft to establish an inflation chamber between the balloon membrane and the outer surface of the shaft, wherein the balloon membrane has a central thickness t_{c} in a region midway between the proximal and distal ends of the membrane, with a membrane thickness tₑ at the proximal end of the membrane, and a substantially same membrane thickness tₑ at the distal end of the membrane, and wherein tₑ>t_{c} to form the membrane with a relatively flat inter-contact surface in the midway region, when the balloon is inflated;
advancing the distal end of the elongated catheter shaft to a treatment site; and
pressurizing the inflation chamber of the balloon to inflate the balloon and position the distal end of the catheter shaft.

19. A method as recited in claim 18 wherein the pressurizing step is accomplished manually by an inflation unit.

20. A method as recited in claim 18 wherein the balloon membrane is made of a compliant material.
